(19) United States
(12) Patent Application Publication
(10) Pub. No.: US 2019/0309316 A1
(43) Pub. Date: Oct. 10, 2019

(54) METHOD OF GENERATING STRESS TOLERANT PLANTS OVER-EXPRESSING CARRP1, REAGENTS AND USES THEREOF

(71) Applicant: National Institute of Plant Genome Research., New Delhi (IN)

(72) Inventors: Niranjan Chakraborty, New Delhi (IN); Subhra Chakraborty, New Delhi (IN); Vijay Wardhan, New Delhi (IN); Divya Rathi, New Delhi (IN); Sonika Gupta, New Delhi (IN)

(21) Appl. No.: 15/781,823

(22) PCT Filed: Dec. 7, 2016
(86) PCT No.: PCT/IN2016/050437

§ 371 Date: Jun. 6, 2018

(30) Foreign Application Priority Data
Dec. 7, 2015 (IN) 3983/DEL/15

(51) Int. Cl. C12N 15/82; C07K 14/415

(52) U.S. Cl. CPC C12N 15/8205; C12N 15/8279; C12N 15/8273; C07K 14/415

(57) ABSTRACT

A method of generating stress tolerant plants which heterologously express a protein of amino acid sequence as set forth in SEQ ID NO: 2. Also, provided are stress tolerant plants which are able to resist varied biotic and abiotic environmental stressors.

### CROSS REFERENCE TO RELATED APPLICATIONS

This application is the U.S. National Phase Application of PCT/IN2016/050437, filed Dec. 7, 2016, which claims priority to Indian Patent Application No. 3983/DEL/15, filed Dec. 7, 2015, the contents of such applications being incorporated by reference herein.

### FIELD OF THE INVENTION

The present disclosure relates to the field of plant genetics and plant transformation. There is provided in the instant disclosure a method of making abiotic stress tolerant transgenic plant, and methods thereof.

### BACKGROUND OF THE INVENTION

Plants, as sessile organisms, are endowed with highly sophisticated biological mechanisms to precisely regulate their growth and development in response to environmental risk factors. Among several abiotic stresses, water-deficit or dehydration is the fundamental factor that negatively affects plant productivity resulting in major yield loss of foremost crops worldwide (Altman et. al., Planta 218, 1-14 (2003); Bray, J. Exp. Bot. 55, 2331-2341 (2004), which are incorporated by reference). Dehydration response in plants is a complex phenomenon, and the exact structural and functional modifications caused by dehydration are poorly understood. Plants render dehydration tolerance either by escaping or by maintaining a favourable internal water balance (Oliveira et al., J. Exp. Bot. 55, 2365-2384 (2004); Bhushan et al., Mol. Cell. Proteomics 6, 1868-1884 (2007), which are incorporated by reference). The changes at molecular level are regulated by a number of different, and potentially overlapping, signal transduction pathways (Zhu et al., Plant Cell 14, S165-S183 (2002); Yamaguchi-Shinozaki et al., J. Exp. Bot. 58, 221-227 (2007), which are incorporated by reference). Interestingly, dehydration induces the expression of proteins not specifically related to this stress, but rather to reactions against cell damage (Zivy et al., Plant Physiol. 117, 1253-1263 (1998), which is incorporated by reference). The dehydration-responsive cellular cross-talk is thought to be modulated by a diverse population of secreted proteins, and thus, expression profiling of proteins has become an important tool to investigate various stress-responsive signaling networks.

The analysis of secretome appears to be a fundamental approach to map the quality and quantity of the extracellular proteins, which provides insight into possible biological pathways involved. The secretomes of plants submitted to stress condition usually contain significantly more leaderless secretory proteins (LSPs) than the secretomes of unstressed plants (Rakwal et al., Proteomics 10, 799-827 (2010), which is incorporated by reference). During the past few years, there have been rapid advances in plant secretomics (Djordjevic et al., J. Proteome Res. 7, 4508-4520 (2008); Sanchez et al., Mass Spectrom. Rev. 28, 844-867 (2009); Juegens et al., Biochim. Biophys. Acta 1834, 2429-2441 (2013), which are incorporated by reference). While most research interest in the secreted proteins has been restricted to the biotic stress responses, the study of the effect of abiotic stress remained highly limited. In the recent past, the studies on plant transcriptomics enhanced our understanding of the molecular basis of abiotic stress response (Yamaguchi-Shinozaki et al., J. Exp. Bot. 58, 221-227 (2007); Blum, Austral. J. Agr. Res. 56, 1159-1168 (2005), which are incorporated by reference).

Developmental control of protein expression in differentiated tissues is assumed to mask the identity of proteins that are differentially expressed as part of stress response. Therefore, investigation of stress-responsive proteins in undifferentiated cells, particularly in suspension-cultured cells (SCCs) has potential for: (i) identifying novel proteins related to stress which have not yet been characterized and (ii) recognizing already characterized proteins that have not been identified as stress-responsive proteins.

### SUMMARY OF THE INVENTION

In an aspect of the present disclosure, there is provided a method of generating a stress tolerant transgenic plant, said method comprising: (a) obtaining plant cells; (b) obtaining a DNA construct comprising a polynucleotide fragment encoding a polypeptide having amino acid sequence as set forth in SEQ ID NO: 2, operably linked to a promoter and transforming a host cell with a DNA vector comprising said DNA construct to obtain a recombinant host cell comprising said DNA construct; (c) transforming said plant cells with said recombinant host cell to obtain transformed plant cells; and (d) selecting transformed plant cells and regenerating transgenic plants, wherein said transgenic plants are tolerant to stress compared to wild type non-transformed plants.

In an aspect of the present disclosure, there is provided a method of generating a stress tolerant transgenic plant, said method comprising: (a) obtaining plant cells; (b) obtaining a DNA construct comprising a polynucleotide fragment encoding a polypeptide having amino acid sequence as set forth in SEQ ID NO: 2, operably linked to a promoter; (c) transforming said plant cells with said DNA construct to obtain transformed plant cells; and (d) selecting transformed plant cells and regenerating transgenic plants, wherein said transgenic plants are tolerant to stress compared to wild type non-transformed plants.

In an aspect of the present disclosure, there is provided a stress tolerant transgenic plant, or parts thereof including seeds, capable of heterologous expression of a polypeptide having amino acid sequence as set forth in SEQ ID NO: 2.

In an aspect of the present disclosure, there is provided a polynucleotide fragment encoding a polypeptide having amino acid sequence as set forth in SEQ ID NO: 2 for use in generating stress tolerant transgenic plants.

These and other features, aspects and advantages of the present subject matter will be better understood with reference to the following description and appended claims. This summary is provided to introduce a selection of concepts. This summary is not intended to identify key features or essential features of the claimed subject matter, nor is it intended to be used for to limiting the scope of the claimed subject matter.

### BRIEF DESCRIPTION OF THE ACCOMPANYING DRAWINGS

The following drawings form part of the present specification and are included to further illustrate aspects of the present disclosure. The disclosure may be better understood by reference to the drawings in combination with the detailed description of the specific embodiments presented herein.

FIG. 1 depicts the physiochemical changes after exposure to PEG-induced dehydration in callus cultures, in accordance with an embodiment of the present disclosure.

FIG. 2 depicts the dehydration induced morphological and ultrastructural changes in suspension culture, in accordance with an embodiment of the present disclosure.

FIG. 3 depicts the purity evaluation of chickpea secreted fraction, in accordance with an embodiment of the present disclosure.

FIG. 4 depicts the resolution of secreted protein on 1-D and 2-DE gel, in accordance with an embodiment of the present disclosure.

FIG. 5 depicts the dehydration-responsive comparative secretome and the representative 2-DE gels, in accordance with an embodiment of the present disclosure.

FIG. 6 depicts the quantitative analysis of changes in protein expression, in accordance with an embodiment of the present disclosure.

FIG. 7 depicts the characteristic features of secreted proteins and their functional classification, in accordance with an embodiment of the present disclosure.

FIG. 8 depicts the structural and phylogenetic analysis of CaRRP1, in accordance with an embodiment of the present disclosure.

FIG. 9 depicts the subcellular localization and secretion analysis of leaderless protein CaRRP1, in accordance with an embodiment of the present disclosure.

FIG. 10 depicts the transcript analysis of CaRRP1 in response to dehydration stress, in accordance with an embodiment of the present disclosure.

FIG. 11 depicts the determination of CaRRP1 transcript levels by qRT-PCR, in accordance with an embodiment of the present disclosure.

FIG. 12 depicts the CaRRP1 complementation of function of YJL036w in yeast, in accordance with an embodiment of the present disclosure.

FIG. 13 depicts the list of identified secreted proteins in chickpea suspension culture by 2-DE, in accordance with an embodiment of the present disclosure.

FIG. 14 depicts the list of identified secreted proteins in chickpea suspension culture by 1-DE, in accordance with an embodiment of the present disclosure.

### DETAILED DESCRIPTION OF THE INVENTION

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any or more of such steps or features.

### Definitions:

For convenience, before further description of the present invention, certain terms employed in the specification, example and appended claims are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps. The term “including” is used to mean “including but not limited to”. “Including” and “including but not limited to” are used interchangeably.

“Primers” are synthesized nucleic acids that anneal to a complementary target DNA strand by hybridization to form a hybrid between the primer and the target DNA strand, and then extended along the target DNA strand by polymerase activity, e.g., a DNA polymerase. Primer pairs described in the present invention refer to their use for amplification of a target nucleic acid sequence, e.g., by polymerase chain reaction or other conventional nucleic-acid amplification methods.

The term “genetic transformation” refers to a process of introducing a DNA sequence or construct (e.g., a vector or expression cassette) into a cell in which that exogenous DNA is incorporated into a chromosome or is capable of autonomous replication.

The term “transgenic” refers to a cell contains a transgene, or whose genome has been altered by the introduction of a transgene. The term “transgenic” when used in reference to a tissue or to a plant refers to a tissue or plant, respectively, which comprises one or more cells that contain a transgene, or whose genome has been altered by the introduction of a transgene.

The term “transgene” refers to any nucleic acid sequence which is introduced into the genome of a cell by experimental manipulations. A transgene may be an “endogenous DNA sequence,” or a “heterologous DNA sequence” (i.e., “foreign DNA”). A transgene is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

The term “vector” refers to a DNA molecule capable of replication in a host cell and/or to which another DNA segment can be operably linked so as to bring about replication of the attached segment. A plasmid is an exemplary vector.

The term “expression vector” refers to a vector comprising an expression cassette.

The term “polypeptide” and “peptide are used interchangeably for the purposes of the present disclosure.

The term “transformed cell” refers to a cell, the DNA complement of which has been altered by the introduction of an exogenous DNA molecule into that cell.

The term “transgenic plant” refers to a plant or progeny plant of any subsequent generation derived therefrom, wherein the DNA of the plant or progeny thereof contains an introduced exogenous DNA segment not originally present in a non-transgenic plant of the same strain. The transgenic plant may additionally contain sequences which are native to the plant being transformed, but wherein the “exogenous” gene has been altered in order to alter the level or pattern of expression of the gene.

The term “polynucleotide” used in the present invention refers to a DNA polymer composed of multiple nucleotides chemically bonded by a series of ester linkages between the phosphoryl group of one nucleotide and the hydroxyl group of the sugar in the adjacent nucleotide.

### Sequences:

SEQ ID NO: 1 depicts the CaRRP1 cDNA sequence.

SEQ ID NO: 2 depicts the CaRRP1 protein sequence.

SEQ ID NO: 3 depicts the CaRRP1pGEM forward primer.

SEQ ID NO: 4 depicts the CaRRP1pGEM reverse primer.

SEQ ID NO: 5 depicts the CaRRP1pYES2 forward primer.

SEQ ID NO: 6 depicts the CaRRP1pYES2 reverse primer.

SEQ ID NO: 7 depicts the CaRRP1RT forward primer.

SEQ ID NO: 8 depicts the CaRRP1RT reverse primer.

SEQ ID NO: 9 depicts the CaEF1 forward primer.

SEQ ID NO: 10 depicts the CaEF1 reverse primer.

SEQ ID NO: 11 depicts the CaRRP1pENTR forward primer.

SEQ ID NO: 12 depicts the CaRRP1pENTR reverse primer.

In an embodiment of the present disclosure, there is provided a method of generating a stress tolerant transgenic plant, said method comprising: (a) obtaining plant cells; (b) obtaining a DNA construct comprising a polynucleotide fragment encoding a polypeptide having amino acid sequence as set forth in SEQ ID NO: 2, operably linked to a promoter and transforming a host cell with a DNA vector comprising said DNA construct to obtain a recombinant host cell comprising said DNA construct; (c) transforming said plant cells with said recombinant host cell to obtain transformed plant cells; and (d) selecting transformed plant cells and regenerating transgenic plants, wherein said transgenic plants are tolerant to stress compared to wild type non-transformed plants.

In an embodiment of the present disclosure, there is provided a method of generating a stress tolerant transgenic plant, said method comprising: (a) obtaining plant cells; (b) obtaining a DNA construct comprising a polynucleotide fragment encoding a polypeptide having amino acid sequence as set forth in SEQ ID NO: 2, operably linked to a promoter; (c) transforming said plant cells with said DNA construct to obtain transformed plant cells; and (d) selecting transformed plant cells and regenerating transgenic plants, wherein said transgenic plants are tolerant to stress compared to wild type non-transformed plants.

In an embodiment of the present disclosure, there is provided a method as described herein, wherein said plant cells is monocot.

In an embodiment of the present disclosure, there is provided a method as described herein, wherein said plant cells is rice.

In an embodiment of the present disclosure, there is provided a method as described herein, wherein said plant cells is wheat.

In an embodiment of the present disclosure, there is provided a method as described herein, wherein said plant cells is rye.

In an embodiment of the present disclosure, there is provided a method as described herein, wherein said plant cells is millet.

In an embodiment of the present disclosure, there is provided a method as described herein, wherein said plant cells is dicot.

In an embodiment of the present disclosure, there is provided a method as described herein, wherein said plant cells is selected from the group consisting of beans, peas, potato, eggplant, peppers, squash, melons, coffee, citrus, broccoli, turnips, legumes, yams, and apple.

In an embodiment of the present disclosure, there is provided a method as described herein, wherein said polynucleotide fragment sequence is as set forth in SEQ ID NO: 1.

In an embodiment of the present disclosure, there is provided a method as described herein, wherein said polynucleotide fragment is cDNA.

In an embodiment of the present disclosure, there is provided a method as described herein, wherein said transgenic plants tolerant to stress heterologously expresses a polypeptide having amino acid sequence as set forth in SEQ ID NO: 2.

In an embodiment of the present disclosure, there is provided a method as described herein, wherein said transformation is carried out by a process selected from the group consisting of Agrobacterium mediated transformation method, particle gun bombardment method, in-planta transformation method, liposome mediated transformation method, protoplast transformation method, microinjection, and macroinjection.

In an embodiment of the present disclosure, there is provided a method as described herein, wherein said transformation is carried out by Agrobacterium mediated transformation.

In an embodiment of the present disclosure, there is provided a method as described herein, wherein said transformation is carried out particle gun bombardment method.

In an embodiment of the present disclosure, there is provided a method as described herein, wherein said recombinant host cell is E. coli.

In an embodiment of the present disclosure, there is provided a method as described herein, wherein said method results in generation of transgenic plants tolerant to dehydration.

In an embodiment of the present disclosure, there is provided a method as described herein, wherein said method results in generation of transgenic plants tolerant to hypersalinity.

In an embodiment of the present disclosure, there is provided a method as described herein, wherein said method results in generation of transgenic plants tolerant to cold.

In an embodiment of the present disclosure, there is provided a method as described herein, wherein said method results in generation of transgenic plants tolerant to methyl viologen treatment.

In an embodiment of the present disclosure, there is provided a method as described herein, wherein said method results in generation of transgenic plants tolerant to jasmonic acid treatment.

In an embodiment of the present disclosure, there is provided a method as described herein, wherein said method results in generation of transgenic plants tolerant to salicylic acid treatment.

In an embodiment of the present disclosure, there is provided a method as described herein, wherein said polypeptide is present in extracellular space of said transgenic plants.

In an embodiment of the present disclosure, there is provided a method as described herein, wherein said polynucleotide fragment is nuclear genome encoded.

In an embodiment of the present disclosure, there is provided a stress tolerant transgenic plant, or parts thereof including seeds, capable of heterologous expression of a polypeptide having amino acid sequence as set forth in SEQ ID NO: 2.

In an embodiment of the present disclosure, there is provided a stress tolerant transgenic plant as described herein, wherein said polypeptide is encoded by a polynucleotide sequence as set forth in SEQ ID NO: 1.

In an embodiment of the present disclosure, there is provided a stress tolerant transgenic plant as described herein, wherein said plant is a monocot.

In an embodiment of the present disclosure, there is provided a stress tolerant transgenic plant as described herein, wherein said plant is a dicot.

In an embodiment of the present disclosure, there is provided a stress tolerant transgenic plant as described herein, wherein said plant is rice.

In an embodiment of the present disclosure, there is provided a stress tolerant transgenic plant as described herein, wherein said plant is wheat.

In an embodiment of the present disclosure, there is provided a stress tolerant transgenic plant as described herein, wherein said plant is rye.

In an embodiment of the present disclosure, there is provided a stress tolerant transgenic plant as described herein, wherein said plant is millet.

In an embodiment of the present disclosure, there is provided a stress tolerant transgenic plant as described herein, wherein said plant is selected from the group consisting of beans, peas, potato, eggplant, peppers, squash, melons, coffee, citrus, broccoli, turnips, legumes, yams, and apple.

In an embodiment of the present disclosure, there is provided a stress tolerant transgenic plant as described herein, wherein said plant is tolerant to dehydration.

In an embodiment of the present disclosure, there is provided a stress tolerant transgenic plant as described herein, wherein said plant is tolerant to hypersalinity.

In an embodiment of the present disclosure, there is provided a stress tolerant transgenic plant as described herein, wherein said plant is tolerant to cold.

In an embodiment of the present disclosure, there is provided a stress tolerant transgenic plant as described herein, wherein said plant is tolerant to methyl viologen treatment.

In an embodiment of the present disclosure, there is provided a stress tolerant transgenic plant as described herein, wherein said plant is tolerant to jasmonic acid treatment.

In an embodiment of the present disclosure, there is provided a stress tolerant transgenic plant as described herein, wherein said plant is tolerant to salicylic acid treatment.

In an embodiment of the present disclosure, there is provided a stress tolerant transgenic plant as described herein, wherein said plant is produced by a method comprising the steps: (a) obtaining plant cells; (b) obtaining a DNA construct comprising a polynucleotide fragment encoding a polypeptide having amino acid sequence as set forth in SEQ ID NO: 2, operably linked to a promoter and transforming a host cell with a DNA vector comprising said DNA construct to obtain a recombinant host cell comprising said DNA construct; (c) transforming said plant cells with said recombinant host cell to obtain transformed plant cells; and (d) selecting transformed plant cells and regenerating transgenic plants, wherein said transgenic plants are tolerant to stress compared to wild type non-transformed plants.

In an embodiment of the present disclosure, there is provided a stress tolerant transgenic plant as described herein, wherein said plant is produced by a method comprising the steps: (a) obtaining plant cells; (b) obtaining a DNA construct comprising a polynucleotide fragment encoding a polypeptide having amino acid sequence as set forth in SEQ ID NO: 2, operably linked to a promoter; (c) transforming said plant cells with said DNA construct to obtain transformed plant cells; and (d) selecting transformed plant cells and regenerating transgenic plants, wherein said transgenic plants are tolerant to stress compared to wild type non-transformed plants.

In an embodiment of the present disclosure, there is provided a polynucleotide fragment encoding a polypeptide having amino acid sequence as set forth in SEQ ID NO: 2 for use in generating stress tolerant transgenic plants.

In an embodiment of the present disclosure, there is provided a polynucleotide fragment as described herein, wherein said polynucleotide fragment is as set forth in SEQ ID NO: 1.

In an embodiment of the present disclosure, there is provided a polynucleotide fragment as described herein, wherein said polynucleotide fragment is cDNA.

In an embodiment of the present disclosure, there is provided a method as described herein, wherein said DNA construct promoter is a constitutive promoter.

In an embodiment of the present disclosure, there is provided a method as described herein, wherein said DNA construct promoter is 35S promoter.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skills in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary. The example is provided just to illustrate the invention and therefore, should not be construed to limit the scope of the invention.

### Example 1

### Imposition of Water-Deficit Condition in Suspension Cultured Cells (SCCs)

There has been much interest in the ability of eukaryotic cells to regulate their internal water potentials (Epstein et al., Science 210, 399-404 (1980)). Although considerable knowledge of this process in certain algae is available (Macrobbie et al., University of California Press, Berkeley 676-713 (1974), which is incorporated by reference), our understanding of dehydration regulation in higher plants is much more limited. While suspension cultures may be considered as over simplification of the complex mechanisms involved in dehydration response, they represent a highly controllable and homogeneous experimental system. However, there is little information on the degree of dehydration and physio-biochemical characteristics of suspension culture. Sugar alcohols such as mannitol and sorbitol act as common source of carbon and energy and also, as osmotica in response to environmental stress including dehydration (Liu et al., Bot. Bull. Acad. Sci. 43, 107-113 (2002), which is incorporated by reference). On the contrary, PEG is known to mimic dehydration by reducing water availability and modifying the osmotic potential of nutrient solution (Rains, Cambridge University Press, Cambridge, UK 181-196 (1989), which is incorporated by reference). We therefore used PEG as an ideal osmoticum to model dehydration conditions in chickpea suspension culture.

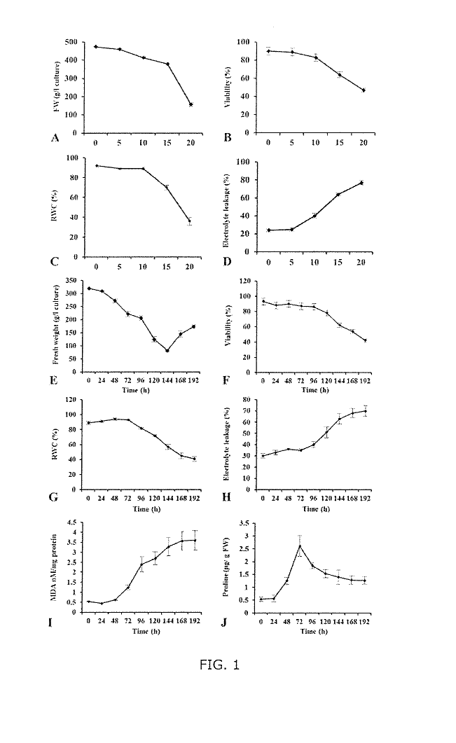

To quantitate the influence of dehydration on the physiology of SCCs, the culture was subjected to various degree of dehydration at varying PEG concentration (0 to 20%). The culture was initially examined for change in fresh weight. No obvious deleterious changes could be observed up to 10% PEG in the media. Subsequent increase in the PEG concentration resulted in sharp decrease in fresh weight (FIG. 1A). Furthermore, viability of the suspension culture demonstrated a positive correlation with the fresh weight measurements. Quantitative determination of cell death indicated that more than 80% cells were viable up to 10% PEG treatment (FIG. 1B). The relative water content (RWC) of the suspension culture was 92% in unstressed condition, which marginally decreased at 10% PEG. However, RWC was drastically decreased to 70% and 36% at 15% and 20% PEG treatments, respectively (FIG. 1C). The electrolyte leakage was found to be maintained at an almost constant level until 5% PEG treatment and showed a marginal increase at 10% PEG. However, a steady increase was observed in 15% and 20% PEG treatments (FIG. 1D). These results indicate that the treatment of 10% PEG imparted moderate dehydration stress, but higher concentrations (15-20%) might cause severe damage (FIG. 1A-D).

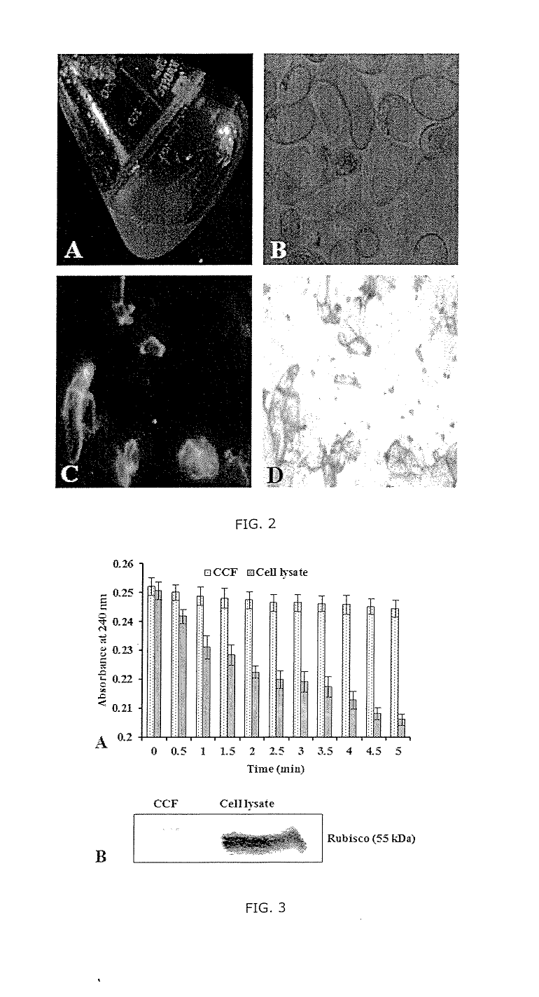

To compute the temporal effect of dehydration on the physiology of culture, 10% PEG treated suspension culture was examined up to 192 h. The morphometric analysis did not show any visible change in the SCCs until 72 h of dehydration (FIG. 2A). The culture was further analyzed microscopically for cellular integrity and homogeneity (FIG. 2B). Approximately 90% of the cells were viable until 72 h of dehydration, when stained with fluorescein diacetate (FDA) and counterstained by Evan's blue (FIG. 2C-D). A time-dependent decline in the fresh weight was observed except for a sudden increase after 144 h (FIG. 1E). This increase might be due to the initiation of second growth cycle of the culture. Cell viability was found to be 86% until 96 h whereas the cell death increased promptly during 120-192 h (FIG. 1F). RWC was found to correlate closely with the growth measurements. There were no significant changes in the RWC during the first 48 h of dehydration. However, a marginal increase in RWC was observed around 48-72 h followed by a gradual decline during later stages (FIG. 1G). A constant level of electrolyte leakage was maintained up to 72 h, but it increased linearly with successive time points (FIG. 1H). Furthermore, lipid peroxidation in terms of MDA accumulations displayed maximum maintenance of cell membrane integrity up to 48 h, portraying a mild stress on the cultured cells. However, the damage aggravated at later stages of 96-120 h (FIG. 1I). Increased levels of compatible solutes after dehydration challenge are common in both intact plants and callus suspension culture (Aspinall et al., Aust. J. Biol. Sci. 26, 45-56 (1973); Bressan et al., Plant Physiol. 80, 938-945 (1986), which are incorporated by reference). Therefore, proline accumulation in the suspension culture was determined across different time points (FIG. 1J). Upon dehydration, proline accumulation curve displayed a peak at 72 h but dropped afterwards. The increase in proline accumulation might cause probable osmotic adjustment, which was also reflected in the increase in RWC during that period.

The purity of the secretome was examined by catalase assay as an intracellular marker. The secreted proteins from dehydration treated suspension culture did not show any significant catalase activity, while proteins prepared from the calli showed high activity (FIG. 3A). We also examined presence of Rubisco, a nonsecretory protein, which could possibly contaminate the secreted proteins. Protein samples were separated by SDS-PAGE and probed with anti-Rubisco antibody. Rubisco was detected in the proteins extracted from calli but not in the secreted fraction (FIG. 3B), indicating that the fraction was essentially free from intracellular proteins.

### Example 2

### Identification of Dehydration-Responsive Secreted Proteins

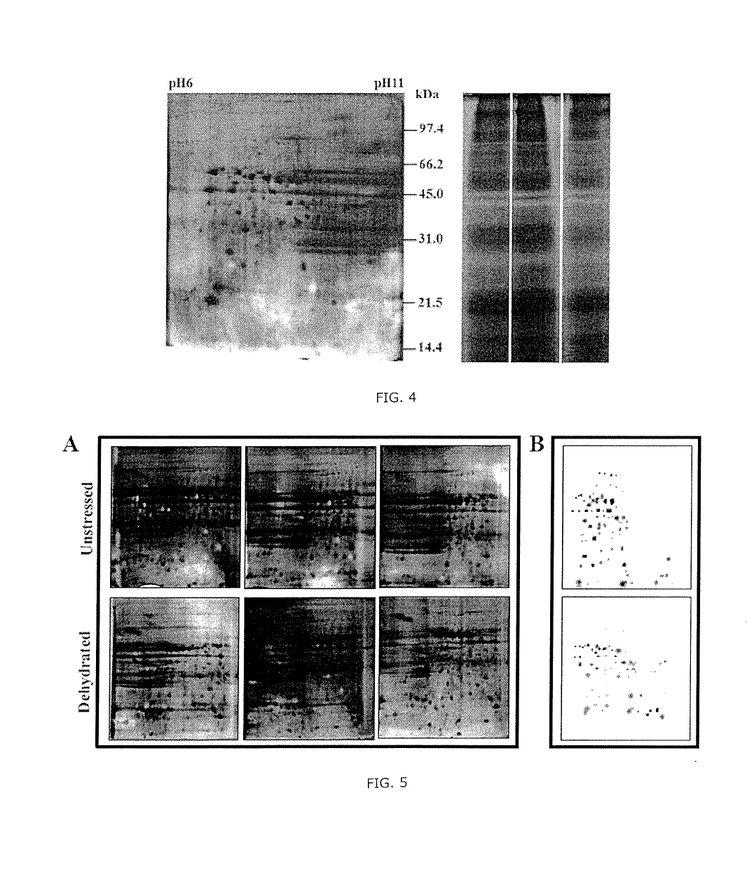

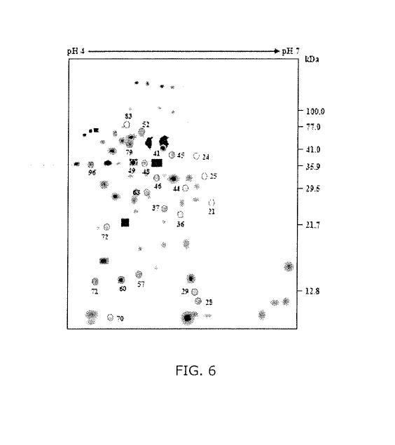

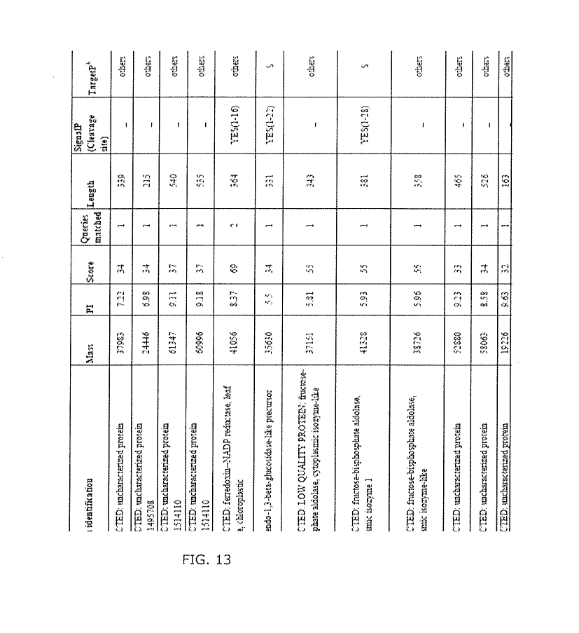

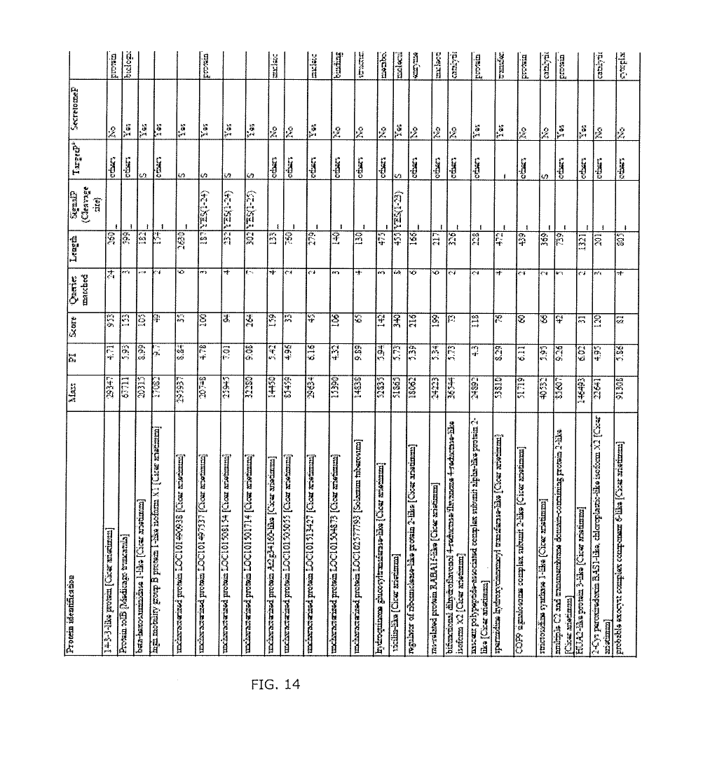

It has been frequently observed that dehydration causes simultaneous activation of specific protein synthesis and inhibition of the synthesis of some constitutive cellular proteins (Scolnik et al., Plant Physiol. 96, 291-296 (1991), which is incorporated by reference). The expression of dehydration-responsive proteins in the suspension culture was thus monitored using 1- and 2-DE analyses. We attempted to resolve the proteins initially onto a basic pH range (pH 6-11) (FIG. 4), which exhibited poor resolution. Henceforth, the proteins were separated onto a pH range of 4-7 (FIG. 5). While 110 spots were detected in unstressed condition, 104 spots could be classified as of high quality (Table 1). After an automated spot detection, the protein spots were checked manually in order to eliminate any possible artifacts such as gel background or streaks. The spot densities were normalized against the total density present in the respective gel to overcome the experimental errors. To make comparison between the stressed and unstressed samples, a second level matchset was created (FIG. 6). The intensity of spots was normalized to that of landmark proteins used for internal standardization. The filtered spot quantities from the higher level matchset were assembled into a data matrix that consisted of 106 spots indicating change in intensity for each spot. The data revealed that nearly 95% of the spots on the reference gels were of high quality, reflecting the reproducibility of the experimental replicates (Table 1). A total of 100 protein spots were reproducibly detected across unstressed and dehydration conditions. Quantitative image analysis revealed a total of 24 protein spots that changed their intensities significantly by more than 2.5-fold. While most spots showed quantitative changes, several spots also showed qualitative changes. Most of these differential spots were low abundant, out of which 8 spots could be processed for downstream analysis. The MS/MS analysis could identify 13 proteins, which are listed in FIG. 13. The secreted proteins were also resolved on 12.5% SDS-PAGE, and gel slices were subjected to MS/MS analysis. Using 1-DE approach, 202 proteins were identified, which would have been largely underrepresented utilizing 2-DE based separation alone (FIG. 14).

### Example 3

### Physicochemical Properties of the Dehydration-Responsive Proteins

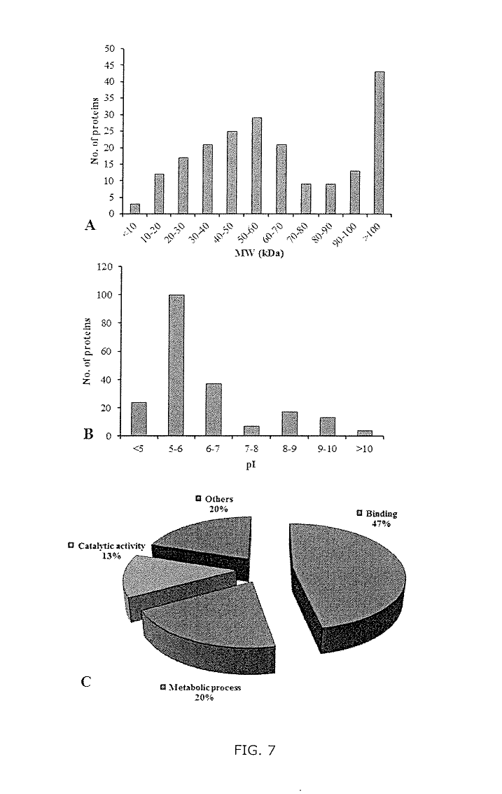

The 202 dehydration-responsive secreted proteins varied in molecular weight with significant increase in the secretion of high molecular weight proteins. In stressed as well as unstressed conditions, maximum number of the identified secreted proteins has relatively high molecular weights in the range >100 kDa (FIG. 7A). However, the isoelectric points (pIs) of the proteins ranged from 4 to 12 with maximum proteins in pI range 5-6 (FIG. 7B). The secretion of acidic proteins was found to be induced by dehydration that accounted for 124 proteins in the pI range of 4 to 6. Many secreted proteins had a pI of more than 8, which corroborate the findings of previous report (Bayer et al., Proteomics 6, 301-311 (2006), which is incorporated by reference).

### Example 4

### Localization Prediction of Dehydration-Responsive Secreted Proteins

Differentially regulated secreted proteins were examined to predict their locations using TargetP program (www.cbs.dtu.dk/services/TargetP). Of the identified proteins, 43 were predicted to be secreted with signal peptide sequences (FIG. 13 and FIG. 14). These proteins were also queried using SignalP 3.0 program, which is a web-based software containing two different algorithms (SignalP-NN and SignalP-HMM). We selected only the proteins harboring a signal peptide by both prediction algorithms (Brunak et al., Protein Eng. Des. Sel. 17, 349-356 (2004), which is incorporated by reference), which accounted for 32 proteins (FIG. 13 and FIG. 14). Proteins predicted to have signal peptide were cross-examined using ScanProsite, with PS00014 as a scan pattern to determine if the proteins were likely to be retained in the endoplasmic reticulum. Only five such proteins containing ER retention motif were excluded from the list of secreted proteins.

Glycosylphosphatidylinositol (GPI) linked proteins are secreted via the ER and Golgi apparatus to the extracellular space. In this study, no proteins were predicted to contain GPI anchors when examined with big-PI Plant Predictor program. There have been many reports of the existence of non-classical proteins in the extracellular space. Protein sequences were thus analyzed on the neural network server SecretomeP. The analysis predicted 63 LSPs (NN-scores >0.5; FIG. 13 and FIG. 14). Further, a critical review of literature, based on independent and orthogonal techniques, supports the presence of 81 of the identified proteins in the secretome. Taken together, 190 proteins that account for more than 90% were confirmed to be localized in the secretory fraction.

### Example 5

### Dynamic Network of Dehydration-Responsive Secreted Proteins

It is increasingly clear that most environmental signals utilize the extra cellular space (ECS) as a conference point for their communication, albeit the role of secreted proteins in such cross-talk remains largely uninvestigated. To gain an insight into protein secretion during stress induced signal transduction, the identified proteins were grouped into 4 functional classes (FIG. 7C, FIG. 13 and FIG. 14). The criteria used to address the localization of the proteins, were based on their presence in other reported cell wall proteomes or secretomes in addition to the predicted biochemical and biological functions. Among the dehydration-responsive proteins in the secretome, the largest percentage was involved in ligands binding proteins (47%), followed by the proteins involved in metabolic process (20%), miscellaneous functions (20%) and catalytic activity (13%).

The communication between the cytoskeleton and the apoplast is one of the most characteristic features of cellular mechanism that allows cells to respond effectively to various extracellular signals, possibly through regulation of ROS (Menzel et al., Plant Physiol. 133, 482-491 (2003), which is incorporated by reference). Induced actin polymer formation is reported during disturbance of ROS homeostasis (Apostolakos et al., Cytoskeleton 69, 1-21 (2012), which is incorporated by reference). Protein kinase is a ubiquitous enzyme in eukaryotes and prokaryotes that catalyzes the transfer of the y-phosphate from ATP to substrate auto-phosphorylation, thus contributing to downstream signaling by producing GTP for the activation of GTP-binding proteins. We observed dehydration induced regulation of protein kinase. Overexpression of protein kinase was previously reported to reduce the accumulation of ROS and provide tolerance against different abiotic stresses (Moon et al., Proc. Natl. Acad. Sci. U.S.A. 100, 358-363 (2003), which is incorporated by reference). Probable serine/threonine-specific protein kinase, xylulose kinase-like, inactive purple acid phosphatase and protein phosphatase are likely candidates that might be involved in the signal transduction network that operates in the apoplast. Subtilisin-like protease, aspartic proteinase, cysteine proteinase and protease inhibitor were previously reported to be regulated differentially under stress conditions (Peng et al., J. Plant Res. 120, 465-469 (2007), which is incorporated by reference). The enzymes related to secondary metabolism were also notable among the list of dehydration-responsive proteins like GMP synthase, an enzyme from the strictosidine biosynthesis pathway. The alkaloids have multiple functions, such as structural support, pigmentation, defense and signaling (Goldberg et al., Annu. Rev. Biochem. 65, 801-847 (1996), which is incorporated by reference). Most of the secondary metabolism related differentially expressed proteins, identified in this study, have been reported in the cell walls of different organisms. Cysteine synthase was earlier reported in cell wall of Medicago (Sumner et al., Phytochemistry 65, 1709-1720 (2004), which is incorporated by reference). Spermidine synthase, serine hydroxymethyltransferase and aldolase were reported in the secondary cell wall of developing xylem tracheary elements (Carol et al., Int. J. Plant Sci. 165, 243-256 (2004), which is incorporated by reference). Hexosaminidase, hydroquinone glucosyltransferase and dolichyl-diphosphooligosaccharide catalyzes an important trafficking step in cell wall modification. Tankyrase-2-like protein is required for synthesis of a variety of cellular constituents including cell wall polymers and glycoproteins. These results suggest that the differentially expressed secreted proteins may utilize the cell wall polysaccharides as a reservoir to produce sugar monomers, which maintain osmotic balance in plants under dehydration conditions.

During stress adaptation, protein degradation is necessary for the removal of abnormal or damaged proteins, and for altering the balance of proteins (Goldberg et al., Annu. Rev. Biochem. 65, 801-847 (1996)). In this study, proteins involved in the degradation pathway were also identified. The 26S proteasome has been reported to be involved in ubiquitin-mediated turnover of misfolded proteins (DiDonato et al., Mol. Cell. Biol. 16, 1295-1304 (1996), which is incorporated by reference). Since intact proteins are less sensitive to oxidation than misfolded proteins, protein chaperones are usually upregulated in response to various stresses. Different chaperones have been documented to play complementary and sometimes overlapping roles in protection of proteins. Many proteins from this class were also identified in the chickpea secretome viz., peptidyl-prolyl cis-trans isomerase, Hsp70 and 10 kDa chaperonin. Furthermore, nodal modulator, which is involved in nodal signaling and subsequent organization of axial structures, was also identified as differentially expressed protein.

The differential regulation of signaling molecules viz., GTP-binding protein and Ran GTPases, in the extracellular space suggests a complex signal transduction network. There are several reports that suggest extensive crosstalk among various environmental stress-responsive pathways (Shinozaki et al., Plant Cell 6, 251-264 (1994); Cheong et al., Plant Physiol. 129, 661-677 (2002), which are incorporated by reference). The PR proteins form a novel class of proteins that play pleiotropic roles, both in abiotic and biotic stresses (Hotta et al., Appl. Biochem. Biotechnol. 120, 169-174 (2005), which is incorporated by reference). Induction of different isoforms of glucanases and thaumatin like-protein, among others, could be attributed to their multiple roles. In unstressed condition, the translation elongation factor (EF-Tu) catalyzes the GTP-dependent binding of the aminoacyl-tRNA to the ribosome during the elongation phase of protein synthesis. However, EF-Tu can act as a molecular chaperone during stress and might be involved in protein folding and protection (Richarme et al., J. Biol. Chem. 273, 11478-11482 (1998), which is incorporated by reference).

### Example 6

### Stress-Induced Non-Classical Secretion

The significance of non-classical secretion in plants, and the possible functions of leaderless secretory proteins (LSPs) are largely unknown. Most of the LSPs are related to stress response, and to explain their secretion, a number of alternate secretion mechanisms have been anticipated (Seedorf et al., Annu. Rev. Cell Dev. Biol. 24, 287-308 (2008); Bissell, et al., Nat. Rev. Mol. Cell Biol. 10, 228-234 (2009), which are incorporated by reference). Leaderless secretion facilitates rapid release of stress-responsive proteins via Golgi/ER-independent pathway. It also allows a normally cytoplasmic protein to relocate to the ECS where it can perform alternate functions. One of the dehydration-responsive LSPs identified, in this study, is nucleolin, a highly conserved and ubiquitously expressed protein in eukaryotes (Bouvet et al., Trends Cell Biol. 17, 80-86 (2007), which is incorporated by reference). It contains RGG repeats which participate in interactions with other proteins. Although it is highly abundant in the nucleus but often found in the plasma membrane and cytoplasm, where it is involved in numerous cellular processes. Lipid transfer protein is known to bind calcium intracellularly, but its role as an extracellular polypeptide signal has also been proposed (Schechter et al., Eur. J. Biochem. 212, 589-596 (1993), which is incorporated by reference). It is likely that these proteins serve dual roles, both intracellular and extracellular, depending on environmental cues. A recent secretome study in mammalian cells revealed an intracellular cysteine protease as a regulator of non-classical protein secretion (Beer et al., Cell 132, 818-831 (2008), which is incorporated by reference). The quantitative study on Arabidopsis suspension culture confirmed that a relatively high number of LSPs are secreted into the ECS when treated with salicylic acid (Williamson et al., J. Proteome Res. 8, 82-93 (2009), which is incorporated by reference). There are an increasing number of reports of individual proteins or enzyme activities that localize to the apoplast in response to stress despite the fact that such proteins lack signal peptide. We identified different isoforms of ubiquitin, proteasome and endopeptidases that lacked the signal peptide (FIG. 14).

### Example 7

### Proteomic Identification and Cloning of CaRRP1, a Non-Classical Secreted Protein

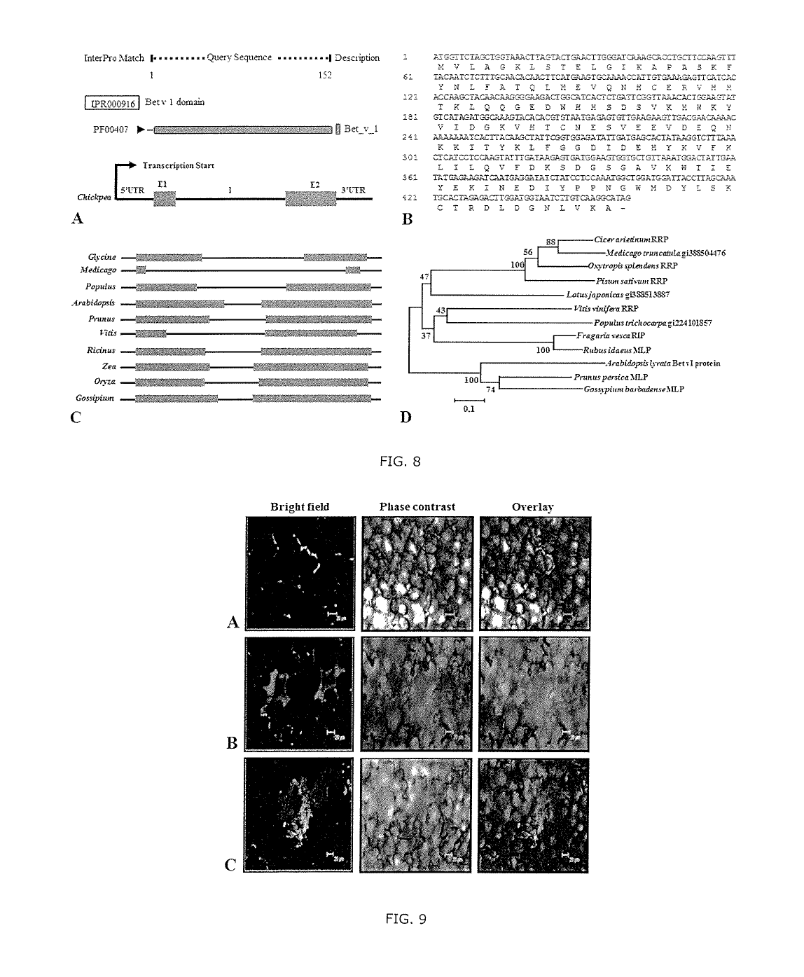

Screening of the secretome led to the identification of a non-classical secreted protein, henceforth designated CaRRP1 (ripening related protein). The domain analysis of CaRRP1 using InterProScan revealed the presence of Bet v 1 family domain (FIG. 8A). The Bet v 1 family proteins are proteins of unknown biological function that were first discovered in the plant latex and found to be upregulated during fruit ripening (Fujimoto et al., Eur. J. Biochem. 258, 794-802 (1998), which is incorporated by reference). Most of these proteins were reported from dicotyledonous plants, and often referred to as cytokinin-specific binding proteins. The RRPs are of interest for several reasons: (i) change in their mRNA expression is accompanied in fruit ripening process; and (ii) the primary structure depicts significant homology to a yeast secretory protein implicated in signal transduction.

### Example 8

### Genomic Organization of CaRRP1 and Phylogenetic Relationship

Genomic sequence comparison revealed the transcript size of CaRRP1 to be 737 bp with coding region of 459 bp, and 43 bp 5′-UTR and 235 bp 3′-UTR. Further, the CaRRP1 coding sequence is interrupted by a single intron (FIG. 8A). The CaRRP1 encodes for a 152 amino acid protein with approximate molecular weight of 17.5 kDa and pI 5.9. The complete nucleotide sequence and deduced amino acid sequence are illustrated in FIG. 8B. The analysis of genomic organization in other taxa revealed that the RRP encoding genes include single intron with varied length ranging from the smallest in Gossypium to the longest in Medicago (FIG. 8C). To determine the evolutionary relationship, phylogenetic analysis was performed using representative RRPs from different taxa. The phylogram displayed two major Bet v 1 evolutionary groups (FIG. 8D). Members of Arabidopsis gene family were found to form distinct clades indicating an evolutionary divergence; however, CaRRP1 closely clustered with proteins from Medicago, possibly because both belong to family Fabaceae.

### Example 9

### Localization of Leaderless CaRRP1

While in silico analysis using SignalP did not identify any clear targeting sequence, SecretomeP neural network programme could recognize the secretion of CaRRP1 via non-classical secretion. To validate the location, the coding region of CaRRP1 was introduced into plant expression vector, harboring YFP reporter gene. The YFP fluorescence of the fusion protein in tobacco leaves was visualized following Agrobacterium-mediated transient expression. A time-dependent expression assay detected the YFP-CaRRP1 in the ECS (FIG. 9). The fluorescent signals were detected in the apoplast at 36 h of agro-infiltration (FIG. 9A), while there was an efflux of signals into the ECS after 48 h (FIG. 9B). To examine further, whether the recombinant protein translocates from cytoplasm to the extracellular space, the leaves were infiltrated with brefeldin A (BFA), an inhibitor of secretion (Driouich et al., Plant Physiol. 114, 401-403 (1997), which is incorporated by reference). The BFA treatment restricted the YFP fusion protein within the cells (FIG. 9C). These results and growing evidence for the unconventional secretion of proteins in non-plant systems support the possibility that alternative export pathways also exist in plants. The LSPs are known intracellular factors involved in various cellular activities in both plants (Vensel et al., J. Proteomics 72, 452-474 (2009), which is incorporated by reference) and mammals (Holmgren et al., Eur. J. Biochem. 267, 6102-6109 (2000), which is incorporated by reference). Interestingly, once these proteins are secreted into the ECS as observed in mammals, they acquire new functions to behave as extracellular signaling molecules (Seedorf et al., Annu. Rev. Cell Dev. Biol. 24, 287-308 (2008)). These findings strongly support the hypothesis that leaderless secretion might be ubiquitous in nature.

### Example 10

### Stress-Induced Expression of CaRRP1

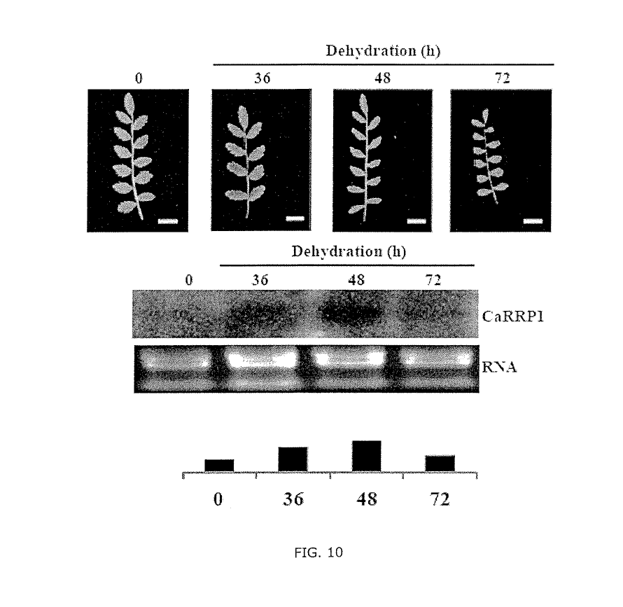

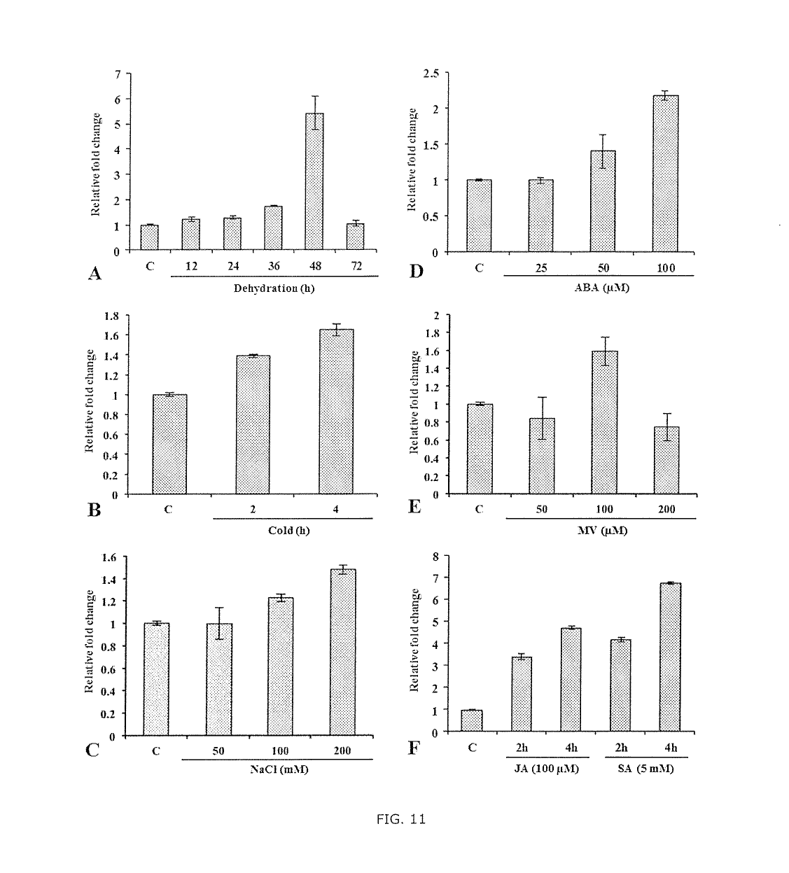

Regulation of gene expression at the transcriptional level plays a crucial role in the development and physiological status of plant. To investigate stress-responsive expression patterns of CaRRP1 in chickpea, we carried out Northern blot analysis. The CaRRP1 transcripts were highly upregulated under dehydration, displaying maximum accumulation at 48 h but dropped thereafter and reached the background level at 72 h (FIG. 10). The quantitative accumulation of transcripts was further examined by qRT-PCR. The results showed that CaRRP1 is responsive to multiple stresses such as dehydration, hypersalinity, cold, and treatment with methyl viologen (MV), jasmonic acid (JA) and salicylic acid (SA). The mRNA signals increased gradually from 12 to 48 h, but decreased at 72 h of dehydration (FIG. 11A). This expression pattern was similar as observed for their mRNA accumulation in response to other stresses such as cold (FIG. 11B), hypersalinity (FIG. 11C) and treatment with ABA (FIG. 11D) indicating that CaRRP1 might participate in abiotic stress response possibly via ABA-dependent pathway. Interestingly, the expression of CaRRP1 displayed increase of 2-fold in response to MV (FIG. 11E) while 4- to 6-fold upon JA and SA treatment (FIG. 11F) indicating its defense responses against diverse biotic stresses.

### Example 11

### Functional Complementation Analysis of CaRRP1 in Yeast

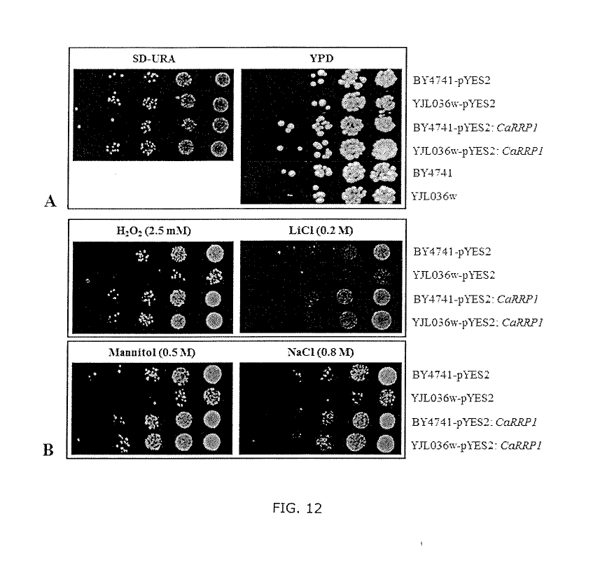

Over the years, complementation analysis in yeast led to the identification of more than 50 proteins involved in protein trafficking in the secretory pathway (Bonifacino, Trends Cell Biol. 24, 3-5 (2014), which is incorporated by reference). YJL036w is a sorting nexin protein involved in proteasome function (Dixon et al., Nat. Rev. Mol. Cell Biol. 3, 919-931 (2002), which is incorporated by reference) and also functions in vesicular transport in yeast. YJL036w lacking yeast strains are defective in protein transport and exhibit aberrations in growth and appearance. To investigate the role of CaRRP1 in protein trafficking, we tested whether it could complement the YJL036w mutant. We used the YJL036w-deficient mutant for the complementation assay and monitored the growth of BY4741 (wild-type), YJL036w mutant and complemented (YJL036w:CaRRP1) strains in presence of various stressors that include 2.5 mM H₂O₂ for oxidative stress, 0.5 M mannitol for osmotic stress, 0.8 M NaCl and 0.2 M LiCl for hypersalinity. No significant difference in growth pattern of transformants, wild-type and mutants was observed either on SD-URA or YPD plates (FIG. 12A). However, the complemented strains grew rapidly and showed more tolerance to stress conditions when compared with the wild-type and mutant strains (FIG. 12B). These results suggest that CaRRP 1 might participate in protein trafficking and restore normal growth in the mutant YJL036w indicating its putative stress-responsive role in secretory pathway.

### Example 12

### Materials and Methods

Maintenance of SSCs and dehydration treatment: Seeds of chickpea (Cicer arietinum L. cv. JG-62) were surface-sterilized using 70% ethanol followed by 0.1% HgCl₂ for 10 min and then rinsed 5-7 times with sterile distilled water. Surface-sterilized seeds were allowed to imbibe water for at least 16 h and kept aseptically in dark. The explants were prepared from root-shoot-cut embryo axes and inoculated on MS media (pH 5.8). The nascent calli were maintained at 25±2° C., under 16 h photoperiod (300 μmol m⁻² s⁻¹ light intensity). The friable embryogenic calli were used to initiate suspension cultures in liquid MS media as described earlier (Gupta et al., J. Proteome Res. 10, 5006-5015 (2011), which is incorporated by reference). Dehydration stress was imposed after second sub-culturing by adding PEG 6000. The unstressed and the stressed cultures were maintained in parallel under same conditions. Secretory fractions of the SCCs, harvested at different time intervals, were used for downstream experiments.

Determination of RWC and cell viability: The SCCs were weighed (fresh weight, FW) after filtration through pre-weighed 0.2 micron membrane. The cells were rehydrated until fully turgid, surface dried and weighed (turgid weight, TW), followed by oven drying at 80° C. for 48 h and reweighed (dry weight, DW). The RWC was calculated by the following formula (Bhushan et al., Mol. Cell. Proteomics 6, 1868-1884 (2007)): RWC (%)=[(FW−DW/TW−FW)×100]. Cell viability of suspension culture was determined and quantified as described earlier²⁰ using Evans blue and FDA.

Proline estimation, evaluation of electrolyte leakage and lipid peroxidation: Free proline content was measured as described earlier. Proline content was determined as per the formula: proline (μg/g FW)=36.6×A₅₂₀×volume/2×FW. Electrolyte leakage was assayed by estimating the ions leaching from the SCCs. Aliquots of 20 ml SCCs in two sets were examined. The first set was kept at 25±2° C. for 4 h and the conductivity (C1) was recorded. The second set was autoclaved followed by recording the conductivity (C2), and electrolyte leakage [1−(C1/C2)×100] was calculated. In a separate experiment, lipid peroxidation was determined in terms of malondialdehyde (MDA) production.

Purification of secreted proteins from suspension culture: The suspension cultures were left untreated or treated with 10% PEG 6000 for 72 h and the secreted proteins were isolated (Gupta et al., J. Proteome Res. 10, 5006-5015 (2011)). In brief, the suspension culture was centrifuged at 3000×g for 5 min followed by filtration using 0.45 μm Durapore membrane (Millipore). The resulting callus culture filtrate (CCF) was concentrated using Amicon Ultra-15 centrifugal filter unit (10 kDa cut-off each; Millipore) until the final protein concentration was >1 mg/ml. Further, the retentate was extracted as described earlier (Peck et al., Mol. Cell. Proteomics 8, 145-156 (2009), which is incorporated by reference). Also, proteins from calli were isolated (Schiltz et al., Plant Physiol. 135, 2241-2260 (2004), which is incorporated by reference) with few modifications. Approximately, 300 mg calli were ground to powder in liquid nitrogen with 0.3% (w/w) polyvinylpolypyrollidone (PVPP) and powdered tissue was homogenized in homogenizing buffer [50 mM Tris-HCl (pH 8.2), 2 mM EDTA, 20% glycerol, 5 mM DTT and 2 mM PMSF]. The proteins were recovered as supernatant by centrifugation at 6000×g for 10 min at4 ° C. (Bhushan et al., J. Proteome Res. 5, 1711-1720 (2006)). Protein samples were allowed to cool at 25±2° C., precipitated with 9 volumes of 100% chilled acetone overnight at −20° C. The precipitates were recovered at 10,000×g for 10 min at 4° C. Protein pellets were washed twice with 80% acetone to remove excess SDS, air-dried and protein concentration was measured using the 2-D Quant Kit (GE Healthcare).

Enzymatic assay of catalase: The catalase activity was determined calorimetrically as described earlier ° C. (Bhushan et al., J. Proteome Res. 5, 1711-1720 (2006)). The reaction mixture was prepared by adding 10 μg protein, in 50 μl, to 940 μl of 70 mM potassium phosphate buffer (pH 7.5). Reaction was initiated by adding 10 μl of H₂O₂ (3% v/v), and decrease in absorbance at 240 nm was monitored for 5 min.

Immunoblot screening: Immunoblotting was carried out by resolving secreted and calli proteins on 12.5% SDS-PAGE followed by electrotransfer onto nitrocellulose membrane (GE Healthcare). The blot was probed with anti-RbcL antibody (AS03037; Agrisera AB) at a dilution of 1:5000 in TBS. Immunoreactive protein was detected by incubation with alkaline phosphatase conjugated anti-rabbit IgG as secondary antibody (Sigma).

Electrophoresis of secreted proteins: The proteins were resuspended in 2-D rehydration buffer [8 M urea, 2 M thiourea, 4% (w/v) CHAPS, 20 mM DTT, 0.5% (v/v) pharmalyte (pH 4-7) and 0.05% (w/v) bromophenol blue]. Isoelectric focusing was carried out with 150 μg protein in 250 μl buffer using 13 cm IPG strips (GE Healthcare) by in-gel rehydration method. Electrofocusing was performed using IPGphor system (GE Healthcare) at 20° C. for 30,000 Vh. In a separate experiment, the proteins were fractionated on 13 cm 1-DE. The electrophoresed proteins were visualized with MS-compatible silver staining (Bio-Rad Laboratories).

Image acquisition and data analysis: Image acquisition was achieved by digitization of gel images with a Bio-Rad FluorS system. Quantitative and qualitative differences between the replicate 2-DE gels were analyzed using PDQuest version 7.2.0 (Bio-Rad Laboratories) followed by generation of reference image (Bhushan et al., Mol. Cell. Proteomics 6, 1868-1884 (2007)). The replicate gels used for making the first level matchset had, at least, a correlation coefficient value of 0.8. In order to compare gels from individual time points, a second level matchset was created. A data matrix of high quality spots was constructed from unstressed and stressed samples for further analysis.

In-gel digestion, mass spectrometry and bioinformatics analysis: The protein spots or lane (sliced into 29 gel pieces each of ˜1.5 mm) were excised and subjected to trypsinolysis (Bhushan et al., J. Proteome Res. 5, 1711-1720 (2006)). The peptides were analyzed using QSTAR Elite mass spectrophotometer (Applied Biosystem) coupled with an on-line Tempo nano-MDLC system. The acquired mass spectra were searched against the chickpea protein sequence available in chickpea genome annotation v.1.0 (Varshney et al., Nat. Biotechnol. 31, 240-246 (2013), which is incorporated by reference) (22893 sequences; 9330989 residues) using Mascot search engine (www.matrixscience.com). Proteins were assigned as identified if the MOWSE score was above the significance level. The function of proteins was assigned using protein function database Pfam (www.sanger.ac.uk/software/Pfam/) or Inter-Pro (www.ebi.ac.uk/interpro/). Further, the criteria used to assign the function of the proteins were based on other reports besides the predicted biochemical and biological functions by GO classification.

The presence and location of signal peptide cleavage sites were predicted by the SignalP 3.0 program (www.cbs.dtu.dk/services/SignalP). Proteins identified by SignalP were re-examined using ScanProsite (au.expasy.org/prosite/). To identify non-classical secreted proteins, proteins lacking N-terminal signal sequences were analyzed by SecretomeP (www.cbs.dtu.dk/services/SecretomeP/). The big-PI Plant Predictor program GPI (mendel.imp.ac.at/gpi/plants/gpi_plants.html) was used to identify potential GPI lipid anchors. The domain analysis was done by NCBI CDD (www.ncbi.nlm.nih.gov).

Isolation of CaRRP1, sequence analyses and construction of phylogram: The cDNA fragment of CaRRP1 was cloned into the pGEM-T vector (Promega) and the sequence identity was determined. The amino acid sequence, molecular weight and isoelectric points of CaRRP1 were obtained from ExPASy. Secondary structures, including the locations of the Bet v 1 domain were determined by InterProScan (www.ebi.ac.uk/interpro/). The SPIDEY (www.ncbi.nlm.nih.gov/spidey) program was used to determine the genomic organization of CaRRP1. The phylogram was constructed from amino acid alignment by neighbor-joining method (www.ebi.uk/Tool/clustalw/) using MEGA software version 5.11, with a bootstrap value of 10060.

Quantitative real-time PCR: The chickpea seedlings were grown in green house, and maintained at 25±2° C. and 50±5% relative humidity under 16 h photoperiod (300 μM m⁻² s⁻¹ light intensity) as described previously. Three-week-old seedlings were independently subjected to dehydration and hypersalinity treatment (50, 100 and 200 mM NaCl). The unstressed seedlings were also maintained in the same green house and tissues were collected every day during the course of the dehydration experiment, and finally pooled to normalize the growth and development effects, if any. ABA (25, 50 and 100 μM), MV (50, 100 and 200 μM), SA (5 mM) and JA (100 μM) treatment were accomplished by spraying solution on the leaflets. The low temperature treatment was given by keeping the seedlings at 4° C. The harvested tissues were instantly frozen in liquid nitrogen and stored at −80° C. Total RNA was isolated from 3-week-old seedlings using the TriPure reagent (Invitrogen). cDNA was prepared using SuperScript® VILO™ cDNA Synthesis Kit (Invitrogen). The qRT-PCR assays were performed with the ABI PRISM 7700 sequence detection system (Applied Biosystems) using SYBR Green PCR Master mix in a final volume of 20 μl including cDNA template and appropriate primers (Table 2).

Localization of CaRRP1: The coding region of CaRRP1 was amplified by PCR using gene-specific primers (Table 2) and cloned into pENTR-D/TOPO followed by recombination into pGWB441, and introduced into Agrobacterium strain GV3101. The cells were infiltrated into tobacco leaves for the transient expression of CaRRP1-YFP fusion protein. The YFP fluorescence was detected with TCS SP2 confocal system (Leica, Germany) and the images were captured 2-3 days after infiltration.

Functional complementation in yeast: The yeast mutant for vesicular transport YJL036w [BY4741; MATa; his3Δ1; leu2Δ0; met15Δ0; ura3Δ0; YJL036w:: kanMX4] and the background strain BY4741 [MATa; his3Δ1; leu2Δ0; met15Δ0; ura3Δ0] were transformed with pYES2 and the recombinant pYES2:CaRRP1 construct independently. Overnight cultures were grown in SD-URA and serial dilutions [OD₆₀₀ ˜0.1] were plated. The plates were incubated at 30° C. for 2 days. Growth assays were performed by using serial dilution of cultures in the respective media containing various stress-inducing chemicals (2.5 mM H₂O₂, 0.5 M mannitol, 0.8 M NaCl and 0.2 M LiCl).

Table 1 depicts the reproducibility of 2-DE gels.

| Time | Total no. of spots^{a} | | High quality spots^{b} | | |
|---|---|---|---|---|---|
| point | Average | SE^{c} | Average | SE^{c} | Reproducibility (%) |
| Control | 110 | 1.54 | 104 | 2.02 | 94.54 |
| 72 h | 105 | 2.67 | 100 | 3.24 | 95.23 |
| Total | 215 | — | 204 | — | 94.88 |

^{a}Average number of spots present in three replicate gels of each time point.
^{b}Spots having quality score more than 30 assigned by PDQuest (Ver.7.2.0).
^{c}SE represents standard error for the replicate gels.

Table 2 depicts list of primers.

| Primer name | Forward primer ID | Reverse primer ID |
|---|---|---|
| CaRRP1pGEM | 3 | 4 |
| CaRRP1pYES2 | 5 | 6 |
| CaRRP1RT | 7 | 8 |
| CaEF1 | 9 | 10 |
| CaRRP1pENTR | 11 | 12 |

Genetic transformation of plant: The coding region of CaRRP1 was amplified by PCR using gene-specific primers and cloned into pENTR-D/TOPO followed by recombination into pGWB441. The CaRRP1-EYFP construct in pGWB441 gateway vector under the control of CaMV 35S promoter was used to transform Agrobacterium strain GV3101. The bacteria were grown to stationary phase in liquid culture at 25-28° C., 250 rpm in sterilized YEP (10 g yeast extract, 10 g peptone, and 5 g NaCl per liter) supplemented with spectinomycin (100 mg/ml). Cultures were initiated with 1:100 dilution of primary overnight cultures and grown for roughly 18-20 h. Cells were harvested by centrifugation (5,500×g) for 20 min at room temperature. The harvested cells were re-suspended in inoculation medium [½ strength MS Medium (M-5519; Sigma Chemicals), 5.0% sucrose, 44 nM benzylamino purine (10 ml l⁻¹ of a 1 mg ml⁻¹ stock in DMSO) and 0.008% Silwet L-77] pH adjusted to 5.7 to a final OD₆₀₀ of approximately 2.0. The seedlings of Arabidopsis were transformed by floral-dip method (Clough and Bent (1998) Plant J. 16(6):735-43, which is incorporated by reference).

### Example 13

### Plant Transformation Protocol

The coding region of CaRRP1 was amplified by PCR using gene-specific primers (SEQ ID NO: 11 and SEQ ID NO: 12) and cloned into pENTR-D/TOPO followed by recombination into pGWB441. The CaRRP1-EYFP construct in pGWB441 gateway vector under the control of CaMV 35S promoter was used to transform Agrobacterium strain GV3101. The bacteria were grown to stationary phase in liquid culture at 25-28° C., 250 rpm in sterilized YEP (10 g yeast extract, 10 g peptone, and 5 g NaCl per liter) supplemented with spectinomycin (100 mg ml⁻¹). Cultures were initiated with 1:100 dilution of primary overnight cultures and grown for roughly 18-20 h. Cells were harvested by centrifugation (5,500×g) for 20 min at room temperature. The harvested cells were resuspended in inoculation medium [½ strength MS Medium (M-5519; Sigma Chemicals), 5.0% sucrose, 44 nM benzylamino purine (10 ml l⁻¹ of a 1 mg ml⁻¹ stock in DMSO) and 0.008% Silwet L-77] pH adjusted to 5.7 to a final OD₆₀₀ of approximately 2.0. The seedlings of Arabidopsis were transformed by floral-dip method [Clough and Bent (1998) Plant J. 16(6):735-43.].

### Example 14

### Figure Legends

FIG. 1: Quantitative determination of (A) callus fresh weight, (B) cell viability, (C) RWC and (D) electrolyte leakage was carried out with increased PEG concentration. Comparative estimation of (E) fresh weight, (F) cell viability, (G) RWC, (H) electrolyte leakage, (I) MDA and (J) proline content at 10% PEG. The experiments were accomplished in triplicates (n=3) and average mean values were plotted. Vertical bars in the graphs indicate the S.E.

FIG. 2: (A) Morphological analysis of chickpea suspension culture. (B) Suspension-cultured cells were scored under bright field illumination. (C) Fluorescence image of the cells stained with FDA to monitor cell viability and emitting green fluorescence. (D) Determination of necrosis in the culture by counter staining with Evan's blue dye.

FIG. 3: (A) Determination of catalase-specific activity in cell lysate and secreted fractions. The SCCs were collected after centrifugation at 3,000 g for 10 min at 4° C. and cell lysates were used as positive control. (B) Immunoblot analysis of secreted proteins with anti-RbcL antibody. An aliquot of 50 μg protein was separated by 12.5% SDS-PAGE and electroblotted onto nitrocellulose membrane. Rubisco was detected using alkaline phosphatase conjugated secondary antibody. Vertical bars in the graphs indicate the S.E.

FIG. 4: (A) Secreted proteins were electrofocused on 13 cm IPG strip (pH 4-7), and separated onto 12.5% SDS-PAGE. (B) Alternatively, the proteins were resolved onto 13 cm 1-DE. The gels were stained by Silver Stain Plus® kit and the spots were visualized as described in Methods.

FIG. 5: (A) Equal amounts (150 μg) of protein from unstressed and treated callus culture were resolved by 2-DE. The experiment was carried out in 3 replicate gels. (B) The replicate gels were combined computationally using PDQuest software (version 7.2.0) to generate the reference gel.

FIG. 6: PDQuest software version 7.2.0 was used to assemble the match sets where replicate gels were compared. The higher level match set of the protein spots, detected by 2-DE, was created in silico by combining data from unstressed and dehydrated secretome. The identified secreted proteins are encircled.

FIG. 7: Ranges of molecular weight (A) and pI (B) of proteins identified in the secretome. Most of the proteins identified have pI between 5 and 10 and a wide range of molecular weight distribution. The identified proteins were assigned a putative function using Pfam and InterPro databases and functionally categorized as represented in the pie chart (C). Percentage of proteins in each assigned class was calculated based on the number of assigned proteins over the total proteins.

FIG. 8: (A) Domain analysis of CaRRP1 by InterPro Scan and schematic representation of its exon-intron organization. (B) Coding sequence of CaRRP1 along with deduced amino acids. (C) Genomic organization of CaRRP1 homologs in other plant species. (D) An unrooted phylogenetic tree showing evolutionary relationship of CaRRP1 with its orthologs. The phylogram was generated using the neighbor-joining algorithm of MEGA software, version 5.11. The numerical represents the bootstrap value. Scale bar indicates an evolutionary distance of 0.1 aa substitution per position in the sequence. E, exon; I, intron; and UTR, untranslated regions.

FIG. 9: Localization was carried out in tobacco leaves using Agrobacterium-mediated transient expression without (A, B) and with (C) BFA (10 μg/mL), an inhibitor of secretion. Yellow fluorescence, visible light and merged images were taken from the epidermal cells, infiltrated with Agrobacterium suspension harbouring the constructs encoding CaRRP1-YFP. Bars=20 μm.

FIG. 10: The total RNA from different tissues, as shown in the upper panel, was fractionated on 1% (w/v) agarose gel, blotted onto nylon membrane, and probed with ³²P-labeled 0.459 kb CaRRP1 cDNA. Ethidium bromide-stained rRNAs (bottom section) shows uniform loading and RNA quality. The graphs are indicative of the extent of expression in terms of band density of CaRRP1.

FIG. 11: Stress induced transcript accumulation was quantified in response to (A) dehydration, (B) cold, (C) NaCl, (D) ABA, (E) MV and (F) JA and SA. Relative fold change in mRNA level is shown on Y-axis. The internal standard EF1α was used for normalizing the qRT-PCR data. The experiments were accomplished in triplicates (n=3). Vertical bars in the graphs indicate the S.E.

FIG. 12: (A) Growth of wild-type (BY4741), BY4741:pYES2, YJL036w and complemented (YJL036w: CaRRP1) strains in unstressed condition, (B) in presence of 2.5 mM H₂O₂ and 0.2 M LiCl (upper panels), 0.5 M mannitol and 0.8 M NaCl (lower panels) in serial dilutions (10 to 10⁻⁵).

### Discussion

Overall, the present disclosure provides a method of producing transgenic plants which are tolerant to stressors such as dehydration, hypersalinity, cold, and chemical stressors such as salicylic acid. These plants are better suited to survive environmental stress conditions, which otherwise may be detrimental to the life of non-transgenic plants. Such plants are of economic significance as environmental stress factor can wreak havoc on plants, which are otherwise agronomically important. Also provided are constructs, vectors, and recombinant cells for facilitation of generating said stress tolerant transgenic plants.

1.-18. (canceled)

19. A method of generating a stress tolerant transgenic plant, said method comprising:
obtaining plant cells;
obtaining a DNA construct comprising a polynucleotide fragment encoding a polypeptide having amino acid sequence as set forth in SEQ ID NO: 2, operably linked to a promoter and transforming a host cell with a DNA vector comprising said DNA construct to obtain a recombinant host cell comprising said DNA construct;
transforming said plant cells with said recombinant host cell to obtain transformed plant cells; and
selecting transformed plant cells and regenerating transgenic plants, wherein said transgenic plants are tolerant to stress compared to wild type non-transformed plants.

20. The method as claimed in claim 19, wherein said method comprises:
obtaining plant cells;
obtaining a DNA construct comprising a polynucleotide fragment encoding a polypeptide having amino acid sequence as set forth in SEQ ID NO: 2, operably linked to a promoter;
transforming said plant cells with said DNA construct to obtain transformed plant cells; and
selecting transformed plant cells and regenerating transgenic plants, wherein said transgenic plants are tolerant to stress compared to wild type non-transformed plants.

21. The method as claimed in claim 19, wherein said plant cells is selected from the group consisting of monocot, and dicot.

22. The method as claimed in claim 21, wherein said plant cells is selected from the group consisting of rice, wheat, rye, millet, beans, peas, potato, eggplant, peppers, squash, melons, coffee, citrus, broccoli, turnips, legumes, yams, and apple.

23. The method as claimed in claim 19, wherein said polynucleotide fragment sequence is as set forth in SEQ ID NO: 1.

24. The method as claimed in claim 20, wherein said polynucleotide fragment sequence is as set forth in SEQ ID NO: 1.

25. The method as claimed in claim 19, wherein said transgenic plants tolerant to stress heterologously expresses a polypeptide having amino acid sequence as set forth in SEQ ID NO: 2.

26. The method as claimed in claim 20, wherein said transgenic plants tolerant to stress heterologously expresses a polypeptide having amino acid sequence as set forth in SEQ ID NO: 2.

27. The method as claimed in claim 19, wherein said transformation is carried out by a process selected from the group consisting of Agrobacterium mediated transformation method, particle gun bombardment method, in-planta transformation method, liposome mediated transformation method, protoplast transformation method, microinjection, and macro injection.

28. The method as claimed in claim 20, wherein said transformation is carried out by a process selected from the group consisting of Agrobacterium mediated transformation method, particle gun bombardment method, in-planta transformation method, liposome mediated transformation method, protoplast transformation method, microinjection, and macroinjection.

29. The method as claimed in claim 19, wherein said transformation is carried out by Agrobacterium mediated transformation method.

30. The method as claimed in claim 19, wherein said recombinant host cell is Agrobacterium.

31. A stress tolerant transgenic plant, or parts thereof including seeds, capable of heterologous expression of a polypeptide having amino acid sequence as set forth in SEQ ID NO: 2.

32. The transgenic plant as claimed in claim 28, wherein said polypeptide is encoded by a polynucleotide sequence as set forth in SEQ ID NO: 1.

33. The transgenic plant as claimed in claim 28, wherein said plant is selected from the group consisting of monocot, and dicot.

34. The transgenic plant as claimed in claim 30, wherein said plant is selected from the group consisting of rice, wheat, rye, millet, beans, peas, potato, eggplant, peppers, squash, melons, coffee, citrus, broccoli, turnips, legumes, yams, and apple.

35. A polynucleotide fragment encoding a polypeptide having amino acid sequence as set forth in SEQ ID NO: 2 for use in generating stress tolerant transgenic plants.

36. The polynucleotide fragment as claimed in claim 32 having sequence as set forth in SEQ ID NO: 1.


SEQUENCE LISTING (12 sequences)

SEQ ID NO: 1 - DNA, 459 bp, artificial sequence
  CaRRP1 cDNA
atggttctagctggtaaacttagtactgaacttgggatcaaagcacctgcttccaagttttacaatctctttgcaacacaacttcatgaagtgcaaaaccattgtgaaagagttcatcacaccaagctacaacaaggggaagactggcatcactctgattcggttaaacactggaagtatgtcatagatggcaaagtacacacgtgtaatgagagtgttgaagaagttgacgaacaaaacaaaaaaatcacttacaagctattcggtggagatattgatgagcactataaggtctttaaactcatcctccaagtatttgataagagtgatggaagtggtgctgttaaatggactattgaatatgagaagatcaatgaggatatctatcctccaaatggctggatggattaccttagcaaatgcactagagacttggatggtaatcttgtcaaggcatag

SEQ ID NO: 2 - protein, 152 aa, artificial sequence
  CaRRP1 protein
MVLAGKLSTELGIKAPASKFYNLFATQLHEVQNHCERVHHTKLQQGEDWHHSDSVKHWKYVIDGKVHTCNESVEEVDEQNKKITYKLFGGDIDEHYKVFKLILQVFDKSDGSGAVKWTIEYEKINEDIYPPNGWMDYLSKCTRDLDGNLVKA

SEQ ID NO: 3 - DNA, 29 bp, artificial sequence
  CaRRP1pGEM forward primer
cgcggatccatggttctagctggtaaact

SEQ ID NO: 4 - DNA, 35 bp, artificial sequence
  CaRRP1pGEM reverse primer
ataagaatgcggccgcctatgccttgacaagatta

SEQ ID NO: 5 - DNA, 29 bp, artificial sequence
  CaRRP1pYES2 forward primer
cgcggatccatggttctagctggtaaact

SEQ ID NO: 6 - DNA, 35 bp, artificial sequence; identical to SEQ ID NO: 4
  CaRRP1pYES2 reverse primer

SEQ ID NO: 7 - DNA, 25 bp, artificial sequence
  CaRRP1RT forward primer
cacctgcttccaagttttacaatct

SEQ ID NO: 8 - DNA, 24 bp, artificial sequence
  CaRRP1RT reverse primer
tctttcacaatggttttgcacttc

SEQ ID NO: 9 - DNA, 20 bp, artificial sequence
  CaEF1 forward primer
tccaccacttggtcgttttg

SEQ ID NO: 10 - DNA, 18 bp, artificial sequence
  CaEF1 reverse primer
cttaatgacaccgacagc

SEQ ID NO: 11 - DNA, 26 bp, artificial sequence
  CaRRP1pENTR forward primer
caccatggttctagctggtaaactta

SEQ ID NO: 12 - DNA, 26 bp, artificial sequence
  CaRRP1pENTR reverse primer
tgccttgacaagattaccatccaagt